(19) United States
(12) Patent Application Publication
(10) Pub. No.: US 2005/0113332 A1
(43) Pub. Date: May 26, 2005

(54) Compositions and methods for treating an arthritic condition

(76) Inventors: Ronenn Roubenoff, Sharon, MA (US); Jacob Selhub, Brookline, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/980,364

(22) Filed: Nov. 2, 2004

Related U.S. Application Data
(63) Continuation of application No. 10/020,634, filed on Dec. 14, 2001.
(60) Provisional application No. 60/255,600, filed on Dec. 14, 2000.

(51) Int. Cl. A61K 31/714; A61K 31/525; A61K 31/205

(52) U.S. Cl. 514/52; 514/251; 514/554; 514/78

(57) ABSTRACT

The present invention features compositions and methods for the treatment of an arthritic condition. The compositions contain a reduced folate compound and a cobalamin compound.

This application is a continuation of U.S. patent application Ser. No. 10/020,634, which claims priority to U.S. provisional application 60/255,600, filed on Dec. 14, 2000, and the entire contents of each is hereby incorporated by reference.

### GOVERNMENT RIGHTS

The development of this invention was made with governmental support under Cooperative Agreement 58-1950-9-001 awarded by the United States Department of Agriculture. The government may have certain rights in the invention.

### FIELD OF THE INVENTION

The present invention makes available compositions and methods for treatment of osteoarthritis.

### BACKGROUND OF THE INVENTION

Arthritis is a common form of joint disease. For example, one form of arthritis, osteoarthritis (“OA”), afflicts countless millions of people worldwide, including more than 50 million Americans.

Osteoarthritis is generally considered to be due to “wear and tear” of the joints leading to damage of the joint surfaces that cause pain on movement. There are many factors influencing its development, including age, family history of OA, and previous damage to the joint through injury or surgery. Symptoms in OA include hard, bony swelling of the joints, and a gritty feeling, or even noise (called crepitus) when the joint is moved. Diagnosis is usually made by a physician, who makes a clinical evaluation of the subject. If a particular joint becomes worse, a physician may arrange X-rays and blood tests in order to confirm the diagnosis.

The present pharmacological management of OA is aimed mainly at symptom relief, through the use of acetaminophen and other analgesics and anti-inflammatory drugs. Non-steroidal anti-inflammatory drugs (NSAIDs) are commonly used, and there are at least 20 of these drugs on the market. NSAIDs are related to aspirin, and they have similar effects but with different duration of action and side effects. For the most part the goal of treatment is to keep the subject mobile and active. However, NSAIDs only treat symptoms, and do not change the progression of the disease. Additionally, all NSAIDs may cause stomach upset, which can sometimes lead to ulcers.

### SUMMARY OF THE INVENTION

The invention provides a safe, well-tolerated composition for treating arthritic conditions. The composition includes a reduced folate compound or a folate compound and confers a clinical benefit upon those afflicted with arthritic conditions such as osteoarthritis or rheumatoid arthritis by reducing the severity or alleviating one or more symptoms of the condition. For example, osteoarthritis of any articulating joint, e.g., the knee, hip, spine, or hand, is treated using the approach described herein.

The composition contains a reduced folate compound and a cobalamin compound in amounts sufficient to exert a chondroprotective effect. Preferably, the composition does not contain acetaminophen. Chondroprotective effects include a reduction in joint pain, improvement in joint mobility, or a reduction in cartilage degradation. The term “cobalamin” refers to vitamin B 12 or vitamers thereof. Vitamers of B12 include derivatives of the vitamin that function a cofactor in folate or reduced folate-mediated methylations.

As is well known in the art, the term “folate” refers to folic acid or a salt thereof. A “reduced folate” is distinguished from a “folate” in that at least one degree of unsaturation is removed. Examples of reduced folates or reduced folic acids include dihydrofolic acid, dihydrofolate, tetrahydrofolic acid, and tetrahydrofolate. Preferably, the composition does not comprise folic acid or a salt thereof. The reduced folate compound is at least 10% more biologically active than an non-reduced (or oxidized) form of folic acid or a salt thereof. Preferably, the reduced folate compound is at least 25% more active, more preferably at least 50% more active, and most preferably at least 100% more active than a non-reduced form of folic acid or a salt thereof. Biological activities of a folate compound include chondroprotection, a reduction in the symptoms of osteoarthritis, e.g., pain or impairment of movement, and methylation capacity.

Reduced folate compounds include 5-formyl tetra hydrofolate, 5-methyl tetrahydrofolate, (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, 5-formimino-(6S)-tetrahydrofolic acid, (6R,S)-tetrahydrofolic acid, 5-methyl-(6R,S)-tetrahydrofolic acid, 5-formyl-(6R,S)-tetrahydrofolic acid, 10-formyl-(6R,S)-tetrahydrofolic acid, 5,10-methylene-(6R,S)-tetrahydrofolic acid, 5,10-methenyl-(6R,S)-tetrahydrofolic acid, and 5-formimino-(6R,S)-tetrahydrofolic acid, or salts thereof.

The invention also includes a composition which contains a reduced folate (or folic acid or a salt thereof), a cobalamin compound, and a betaine compound. The amount of reduced folate (or folic acid or a salt thereof) to achieve a beneficial clinical effect using the folic acid/cobalamin/betaine composition is less than that required if folic acid or its salt was administered in the absence of betaine.

The invention includes methods of treating and arthritic condition such as osteoarthritis and methods of alleviating a symptom of an arthritic condition by administering to a mammal a composition containing a reduced folate compound. Optionally, a cobalamin compound and/or a betaine compound is also administered. Preferably, acetaminophen is not co-administered. The mammal to be treated has been diagnosed as suffering from or at risk of developing an arthritic condition such as osteoarthritis. Preferably, the mammal is not been diagnosed as suffering from depression or Alzheimer's Disease.

The ratio of reduced folate to B12 in the composition ranges from 2.5:1 to 125:1. For example, the ratio is approximately 50:1. The composition contains 0.01 mg to 500 mg of a reduced folate compound. Optionally, the composition contains 0.0002 mg to 10 mg of a cobalamin compound and/or 50 mg to 20,000 mg of a betaine compound. A daily dose of compound includes 0.01 mg to 500 mg of a reduced folate compound. 0.0002 mg to 10 mg of a cobalamin compound and/or 50 mg to 20,000 mg of a betaine compound may be coadministered. A reduced folate compound is preferably administered at a dose of 0.1-5 mg/day or 0.1-50 mg/day; a cobalamin compound is preferably administered at a dose of 0.002-1 mg/day; and, a betaine compound is preferably administered at a dose of 500-2000 mg/day. Reduced folate is administered in the presence or absence of cobalamin or betaine. Folic acid is preferably administered at a dose of 0.1 mg to 5 mg/day. Daily doses refer to amounts administered to an average adult human subject.

The compounds are administered simultaneously or sequentially; they are administered by the same or different route. Administration may be parenteral. Preferably, administration is oral.

The dose of a reduced folate compound is greater than the recommended daily requirement (RDA). For example, the dose is greater than 200% of the recommended daily allowance (RDA) established for healthy adults by the Food and Nutrition Board; the highest daily dietary amount of folic acid recommended is 2.0 mg for healthy adults. No recommendations have been made for reduced folate compounds. The doses described herein are safe for adult humans.

Optionally, the compositions contain or are coadministered with an NSAID, e.g., aspirin, ibuprofen, ketoprofen, naproxen, diclofenac, or diflunisal; a disease-modifying anti-rheumatic drug, e.g, gold, hydroxychloroquinone, penicillamine, or sulfasalazine; an immunosuppressant, e.g., methotrexate, azathioprine, or cyclophosphamide; or a corticosteroid, e.g., prednisone or methylprednisolone. In some embodiments, the reduced folate compound is administered in the absence of an NSAID such as acetaminophen.

The compounds for therapeutic administration is substantially pure. A compound is “substantially pure” when it is in a preparations that is at least 60% by weight (dry weight) the compound of interest. Preferably, the preparation is at least 75%, more preferably at least 90%, and most preferably at least 99%, by weight the compound of interest. Purity is measured by any appropriate standard method, e.g., by column chromatography, polyacrylamide gel electrophoresis, or HPLC analysis. For example, the compounds are isolated from natural sources or chemically synthesized.

Advantages of the compositions and methods described herein include safety and minimal side effects compared to arthritis medications currently in use. Thus, the reduced folate compositions can be administered over longer periods of time without adverse symptoms such as gastrointestinal distress (which is often associated with administration of folic acid). For example, administering reduced folate rather than folic acid leads to an improved clinical condition at a lower dose than would be required for a similar effect with folic acid. The reduced folate preparation described herein is better absorbed than the currently used medicament for treatment of osteoarthritis, s-adenosylmethionine (SAM), which often causes gastrointestinal distress because >90% of the compound remains in the intestine.

Unless defined otherwise, all technical and scientific terms used herein have the same meaning as commonly understood by one of ordinary skill in the art to which this invention belongs. Other features and advantages of the invention will be apparent from the following description of the preferred embodiments thereof and from the claims.

### BRIEF DESCRIPTION OF THE DRAWINGS

FIG. 1 is a diagram of the biochemical pathway for SAM generation.

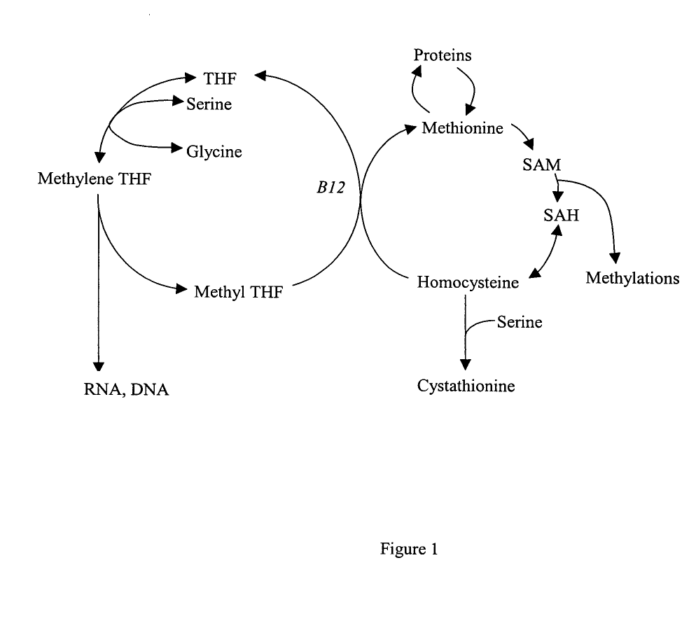

### DETAILED DESCRIPTION

Prior to the invention, the treatment of OA relied largely on symptomatic treatment with NSAIDs. Although NSAIDs help control pain and joint swelling, they do not modify the course of the disease, and joint replacement surgery is often needed. In fact, such surgery has become one of the most commonly performed group of operations in the developed world, with over half a million joint replacements in the U.S. each year. In addition, NSAIDs cause many problems, including gastrointestinal hemorrhage, peptic ulcer disease, gastritis, renal insufficiency, and central nervous system side effects. NSAIDs are a leading cause of iatrogenic complications and hospitalizations, especially in the elderly.

The effect of folate and cobalamin on osteoarthritic hands has been studied (Flynn et al., 1994, J. Am. Coll. Nutr. 13: 351-356). The method described herein, which utilizes reduced folate, is surprisingly more effective in improving the condition of an arthritic joint and in alleviating pain compared to folate (in its oxidized or non-reduced form). In the Flynn reference, patients were treated with 6400 mg “folate” per day or 6400 mg “folate” together with 20 mg cyanocobalamin per day (page 352, right column, lines 8-12). The invention is distinguished from this method because the methods described herein preferably utilize reduced folate rather than “folate” (i.e., oxidized form) or a mixture of folic acids, folate/folacin at various oxidation states. Secondly, the amount of cyanocobalamin used by Flynn et al. is lower (by at least a factor of 20) than the amount of cobalamin compound used in the methods described herein.

Folate Compounds: Reduced Forms and Oxidized Forms

Folic acid (also referred to as “folate”, vitamin B-9, or pteroyl-L-glutamic acid) is a water soluble B vitamin. Folic acid (C₁₉H₁₉N₇0₆) or a salt thereof (“folate”), e.g., sodium folate (C₁₉H₁₈N₇O₆Na) represent the oxidized forms of this compound. Sodium folate or calcium folate is used medicinally in folic acid therapy. Biochemically, folic acid (or folate) functions as a methyl donor after being enzymatically reduced in the body to tetrahydrofolate by the enzyme dihydrofolate reductase. In the absence of a modifier, the term “folate” in the art refers to the oxidized form of the compound. High doses of folic acid or folate can cause flatulence, loss of appetite, and nausea.

Reduced folate compounds differ significantly from the oxidized forms and the advantages associated with the reduced form were surprising. Compared to the oxidized forms (folic acid or folate), the reduced folate compounds have more potent biological activity and are better tolerated (i.e., fewer adverse side effects are observed with administration of reduced folate compounds). In folic acid or folate, the pteridine ring is fully oxidized and there is a sole chiral center. The reduced forms (e.g., tetrahydro forms) have two chiral centers. In addition to the single chiral center of the L-glutamate chain in folic acid, the tetrahydrofolates contain a second stereochemical center at carbon-6. Chemical reduction of folic acid produces a nearly racemic mixture of the two isomers at this position. This is in contrast to the reduced folates found in nature which all consist of a single diastereoisomer, all having the same L-configuration at carbon-6. Both the racemic mixture (R,S) and purified preparations of a diastereomer, e.g., a purified 6S form, are useful to treat osteoarthritis. Administration of a naturally-occurring isomer of a reduced folate compound is preferred.

Nomenclature and Preparation of Reduced Folate Compounds

Conventional terminology is used to designate the isomers as described below and in appropriate text books known to those of ordinary skill in the art. (See, e.g., Principles in Biochemistry, Lehninger (ed.), page 99-100, Worth Publishers, Inc. (1982) New York, N.Y.; Organic Chemistry, Morrison and Boyd, 3rd Edition, Chap. 4, Allyn and Bacon, Inc., Boston, Mass. (1978). The asymmetric a carbon atom of the compound is referred to as a chiral center and can occur in two different isomeric forms. These forms are identical in all chemical and physical properties with one exception, the direction in which they can cause the rotation of plane-polarized light.

In general, naturally occurring compounds which contain a chiral center are only in one stereoisomeric form, either D or L. However, compounds which have two or more chiral centers may be in 2ⁿ possible stereo isomer configurations, where n is the number of chiral centers. These stereo isomers sometimes are designated using the RS system to more clearly specify the configurations of amino acids that contain two or more chiral centers. The isomers of compounds having two chiral centers are known as diastereomers.

The RS system was implemented to avoid ambiguities when a compound contains two or more chiral centers. In general, the system is designed to rank the four different substituent atoms around an asymmetric carbon atom in order of decreasing atomic number or in order of decreasing valance density when the smallest or lowest-rank group is pointing directly away from the viewer. The different rankings are well known in the art. If the decreasing rank order is seen to be clock-wise, the configuration around the chiral center is referred to as R; if the decreasing rank order is counter-clockwise, the configuration is referred to as S. Each chiral center is named accordingly using this system.

If a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers. For example, a process for making methyltetrahydrofolic acid is described in U.S. Pat. No. 5,124,452, and formulations for therapeutic administration of folate compounds, including reduced folate compounds are described in U.S. Pat. Nos. 5,538,734, 5,556,644, and 6,127,370 (all of which are hereby incorporated by reference).

For example, a racemic 6(R,S) mixture of 5-formyl-tetrahydrofolic acid (leucovorin or folinic acid) and the racemic mixture of 5-methyl-tetrahydrofolate as well as purified preparations of the S diastereomer are commercially available. Methods for making the compounds are well known in the art and include chromatographic separation, enzymatic reduction, and fractional crystallization. Reduced folate compounds suitable for treatment of any arthritic condition or pre-arthritic condition include (6R,S)-tetrahydrofolic acid, 5-methyl-(6R,S)-tetrahydrofolic acid, 5-formyl-(6R,S)-tetrahydrofolic acid, 10-formyl-(6R,S)-tetrahydrofolic acid, 5,10-methylene-(6R,S)-tetrahydrofolic acid, 5,10-methenyl-(6R,S)-tetrahydrofolic acid, 5-formimino-(6R,S)-tetrahydrofolic acid, and polyglutamyl derivatives thereof as well as purified 6R or 6S forms of the compounds. For example, purified (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, and 5-formimino-(6S)-tetrahydrofolic acid are used.

SAM and Osteoarthritis

S-Adenosyl Methionine (SAM-e) is active Methionine. Methionine is an essential sulfur-containing amino acid that is converted in the liver and other tissues into S adenosyl methionine (SAM), which is considered to be the activated form of methionine. This happens in the first step of the metabolism of methionine and requires energy in the form of ATP. In this activation of methionine, an adenosyl moiety is transferred from the ATP molecule. SAM synthesis is one of the most potent methyl donors and is involved in many methylation reactions. Its methyl group, which is attached in a sulfonium linkage with high-energy characteristics, may be donated to any of a large number of methyl-group acceptors in the presence of the appropriate enzyme. Once SAM transfers its methyl group, homocysteine and adenosine are produced.

In the presence of reduced folate compounds (e.g., tetrahydrofolate), vitamin B6, vitamin B12, magnesium and a source of methyl groups from either betaine, serine or dimethylglycine (DMG), homocysteine can be recycled back into methionine and subsequently to SAM. This methyl transfer reaction can be done from betaine in the absence of B12 or from methyl-tetrahydrofolate or methyl-tetrahydrofolic acid in the presence of vitamin B12.

Methionine is needed for SAM synthesis and SAM-dependent processes. Homocysteine can replace methionine in the diet when either of two conditions are satisfied: (1) folic acid and cobalamin (vitamin B12) are supplemented, or (2) choline or betaine are supplemented. There are two pathways for production of methionine from homocysteine, one involving folate and cobalamin and the other choline and its catabolites (betaine). The folate (or reduce folate) system or pathway is found in all tissues, whereas the choline/betaine system is found in liver and kidney.

The compositions described herein increase SAM production in the body of the recipient, thereby improving the clinical condition of patients suffering from or at risk of developing osteoarthritis. Reduced folate compounds of the invention have a better safety profile than SAM and commonly-used analgesic compounds with comparable pain-relieving capacity and disease-modifying activity (e.g., chondroprotection). For example, according to the invention, 5-methyltetrahydrofolate and other reduced folate compounds are used to treat OA, because it has a similar biochemical fate to SAM, but is better absorbed and less likely to cause gastrointestinal side effects than SAM.

Betaine Compounds

Betaine is also required for the conversion of homocysteine to methionine, which is then converted to SAM. Although folic acid or folate at high concentrations can lead to adverse side effects, the amount of folate or reduced folate compound required for a beneficial clinical effect is reduced by administering the compounds with cobalamin and betaine.

Administration of Therapeutic Compound

5-methyltetrahydrofolate, in combination with vitamin B12, is a disease-modifying agent in OA. For example, 50 mg 5-methyltetrahydrofolate and 1 mg vitamin B12 per day is administered to reduce joint pain and improve mobility or physical performance. Joint pain is evaluated using methods known in the art, e.g., the Western Ontario-McMaster Arthritis Center questionnaire (WOMAC). Mobility or physical performance is measured by 50 foot walking time and chair stand time. Another index of clinical benefit is methylation capacity. Improvement in a patient's methylation capacity is assessed by plasma, red blood cell (RBC) and lymphocytes levels of SAM, MTHF levels, and extent of DNA methylation. Cartilage degradation is measured using methods known in the art, e.g., x-rays.

The compounds of the invention are administered orally or by other standard routes, e.g., intramuscularly or intravenously. The dose of oral administration of the reduced folate is in the range of 0.1 to 500 mg per day, preferably 0.1 to 50 mg per day. The daily dose may be given in one administration or divided into several (e.g., 2 to 4) dosages in a day.

Reduced folates in conjunction with cobalamin and/or betaine are administered in a pharmaceutically acceptable carrier. An effective amount is an amount of a composition that is required to improve the clinical condition of a patients. An improvement in clinical condition includes a decrease in pain, an increase in mobility, an improvement in methylation capacity, and/or chondroprotection. The effective amount of a composition or compound may depend on a number of factors, including the age, race, and sex of the subject and the progression or severity of the disease and other factors responsible for biologic variability. Though the term effective amount in the present invention is not limited to a particular mechanism of action for a specific composition or compound, an effective amount may be that amount of a composition or compound required to reduce the symptoms of disease (e.g. osteoarthritis) in a subject.

The term pharmaceutically acceptable carrier refers to a molecule or other excipient that can carry or deliver a composition or other active ingredient (e.g. MTHF) to a subject. Suitable pharmaceutically and physiologically acceptable carriers include, but are not limited to, water, salt solutions, alcohols, glycols, vegetable oils, polyethylene glycols, gelatin, lactose, amylose, magnesium stearate, talc, silicic acid, viscous paraffin, perfume oil, fatty acid monoglycerides and diglycerides, pentaerythritol fatty acid esters, hydroxymethyl cellulose, polyvinyl pyrrolidine, etc. The pharmaceutical preparations can be sterilized and, if desired, mixed with auxiliary agents, including lubricants, preservatives, stabilizers, wetting agents, emulsifiers, salts for influencing osmotic pressure, buffers, coloring, flavoring and/or aromatic substances and the like which do not deleteriously interact with the active compound.

Solutions, preferably glycol, oil or alcohol solutions, as well as suspensions, emulsions, or implants can be used for parenteral application. Unit dosages can conveniently be provided in ampoules. Pharmaceutical compositions containing one or more active ingredient (e.g., reduced folate and/or a cobalamin or betaine compound) are formulated in a form suitable for oral use, for example, as tablets, troches, lozenges, aqueous or oily suspensions, dispersible powders or granules, emulsions, hard or soft capsules, or syrups or elixirs. Compositions intended for oral use are prepared according to known methods, and such compositions may contain one or more agents selected from the group consisting of sweetening agents, flavoring agents, coloring agents and preserving agents in order to provide pharmaceutically elegant and palatable preparations. Tablets contain an active ingredient together with a non-toxic pharmaceutically acceptable excipient. Excipients include inert diluents, such as calcium carbonate, sodium carbonate, lactose, calcium phosphate or sodium phosphate; granulating and disintegrating agents, e.g., corn starch, or alginic acid; binding agents, e.g., starch, gelatin or acacia, and lubricating agents, e.g., magnesium stearate, stearic acid or talc. Tablets may be uncoated or enterically coated to delay disintegration and absorption in the gastrointestinal tract and thereby provide a sustained action over a longer period. For example, a time delay material such as glyceryl monostearate or glyceryl distearate is employed.

Formulations for oral use also include hard gelatin capsules in which the active ingredient is mixed with an inert solid diluent, e.g., calcium carbonate, calcium phosphate or kaolin, or soft gelatin capsules in which the active ingredient is mixed with water or an oil medium such as peanut oil, liquid paraffin, or olive oil.

Aqueous suspensions contain the active ingredients in admixture with excipients suitable for the manufacture of aqueous suspensions. Such excipients include suspending agents, e.g., sodium carboxymethylcellulose, methylcellulose, hydroxy-propylmethylcellulose, sodium alginate, polyvinyl-pyrrolidone, gum tragacanth and gum acacia. Dispersing or wetting agents include a naturally-occurring phosphatide, e.g., lecithin, or a condensation product of an alkylene oxide with fatty acids, e.g., polyoxyethylene stearate, or a condensation products of ethylene oxide with long chain aliphatic alcohol, e.g., heptadecaethyleneoxycetanol, or a condensation product of ethylene oxide with partial esters derived from fatty acids and a hexitol such as polyoxyethylene sorbitol monooleate, or a condensation product of ethylene oxide with partial esters derived from fatty acids and hexitol anhydrides, e.g., polyethylene sorbitan monooleate. Aqueous suspensions may also contain one or more preservatives, e.g., ethyl, or n-propyl, p-hydroxybenzoate, one or more coloring agents, one or more flavoring agents, and one or more sweetening agents, such as sucrose or saccharin.

Oily suspensions are formulated by suspending one or more active ingredient in a vegetable oil, e.g., arachis oil, olive oil, sesame oil or coconut oil, or in a mineral oil such as liquid paraffin. Oily suspensions may contain a thickening agent, e.g., beeswax, hard paraffin or cetyl alcohol. Sweetening agents and flavoring agents are added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, e.g., gum acacia or gum tragacanth, naturally-occurring phosphatides, e.g., soybean, lecithin, and esters or partial esters derived from fatty acids and hexitol anhydrides, e.g., sorbitan monooleate, and condensation products of partial esters with ethylene oxide, e.g., polyoxyethylene sorbitan monooleate. Syrups and elixirs may be formulated with sweetening agents, e.g., glycerol, propylene glycol, sorbitol or sucrose. Such formulations may also contain a demulcent, a preservative and flavoring and coloring agents.

The pharmaceutical compositions may be in the form of a sterile injectable aqueous or oleagenous suspension. The suspension is formulated according to methods known in the art. Among the acceptable vehicles and solvents that may be employed are water, Ringer's solution and isotonic sodium chloride solution.

The compounds of the present invention may also be administered in the form of suppositories for rectal administration. These compositions are prepared by mixing the compound, e.g, a reduced folate compound, with a suitable non-irritating excipient which is solid at ordinary temperatures but liquid at the rectal temperature. Melting in the rectum release the drug into surrounding tissues. Such materials include cocoa butter and polyethylene glycols.

The compositions described herein are optionally mixed with or co-administered with other therapeutic agents such as NSAIDs such as ibuprofen and cyclooxygenase inhibitors such as rofecoxib, steroids such as prednisone or dexamethasone,

For oral administration, the compositions are preferably provided in the form of tablets containing 0.01 to 1000 mg of a reduced folate compound, e.g., 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient. The compounds are administered on a regimen of 1 to 4 times per day, e.g., once or twice per day. A therapeutically effective amount is an amount of compound that alleviates one or more symptoms of an arthritic condition or reduces or slows the progression of the disease or condition.

Dosages described above are provided for an average adult human subject. An effective amount of the reduce folate compound is ordinarily supplied at a dosage level of from about 0.0001 mg/kg to about 25 mg/kg of body weight per day. An average adult is given 0.1 mg to 50 mg of reduced folate per day.

The exact dosage and frequency of administration depends on the particular compositions used, the severity of the condition being treated, the age, weight, general physical condition of the particular patient, other medication the individual may be taking as is well known to those skilled in the art and can be more accurately determined by measuring the blood level or concentration of the compositions in the patient's blood and/or the patient's response to the particular condition being treated.

In addition to primates, such as humans, a variety of other mammals can be treated according to the method of the present invention. Mammals including, but not limited to, cows, sheep, goats, horses, dogs, cats, guinea pigs, rats or other bovine, ovine, equine, canine, feline, rodent or murine species are treated. For example, horses such as equine athletes are treated with the compositions to reduce the symptoms of an arthritic condition. Both subjects at risk for an arthritic condition and subjects having symptoms of such a condition are treated using the compositions and methods herein described. For example, subjects are at risk of developing osteoarthritis include subjects with a family history of the disease or members of a population known to have a high incidence of the disease, e.g., the elderly. Subjects with symptoms of osteoarthritis are those with detectable symptoms (i.e. detectable by observation or diagnostic testing) identified by those skilled in the art, e.g., a physician. Treatment includes amelioration of acute symptoms and prevention of the development of osteoarthritic symptoms.

Other embodiments are within the following claims.

1. A method of treating an arthritic condition comprising administering to a mammal a composition comprising a reduced folate compound.

2. The method according to claim 1, wherein said arthritic condition comprises osteoarthritis.

3. The method according to claim 1, wherein the composition comprises 0.01 mg to 500 mg of said reduced folate compound.

4. The method according to claim 1, wherein the composition further comprises a cobalamin compound.

5. The method according to claim 4, wherein the composition comprises of 0.0002 mg to 10 mg of said cobalamin compound.

6. The method of claim 1, wherein said composition does not comprise folic acid.

7. The method of claim 5, wherein said composition further comprises a betaine compound.

8. The method according to claim 7, wherein the composition comprises 50 mg to 20,000 mg of said betaine compound.

9. The method of claim 1, wherein said reduced folate compound is 5-methyl tetrahydrofolate.

10. The method of claim 1, wherein said reduce folate compound is 5-formyl tetra hydrofolate.

11. The method of claim 1, wherein said reduced folate is selected from the group consisting of (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, and 5-formimino-(6S)-tetrahydrofolic acid.

12. The method of claim 1, wherein said reduced folate compound is administered at a dose of 0.1-50 mg/day.

13. The method of claim 1, wherein said reduced folate is administered at a dose of 0.1-5 mg/day.

14. The method of claim 4, wherein said cobalamin compound is administered at a dose of 0.002-1 mg/day.

15. The method of claim 7, wherein said betaine compound is administered at a dose of 500-2000 mg/day.

16. A method of alleviating a symptom of arthritis, comprising administering to a mammal a composition comprising a reduced folate compound, wherein said mammal is diagnosed as suffering from or at risk of developing arthritis.

17. The method of claim 16, wherein said mammal is diagnosed as suffering from or at risk of developing osteoarthritis.

18. The method according to claim 16, wherein the composition comprises 0.01 mg to 500 mg of said reduced folate compound.

19. The method according to claim 16, wherein the composition comprises 0.1 mg to 50 mg of said reduced folate compound.

20. The method according to claim 16, wherein the composition comprises 0.1 mg to 5 mg of said reduced folate compound.

21. The method according to claim 16, wherein the composition further comprises a cobalamin compound.

22. The method according to claim 21, wherein the composition comprises of 0.0002 mg to 10 mg of said cobalamin compound.

23. The method according to claim 21, wherein the composition comprises of 0.002 mg to 1 mg of said cobalamin compound.

24. The method of claim 16, wherein said composition does not comprise folic acid.

25. The method of claim 21, wherein said composition further comprises a betaine compound.

26. The method according to claim 25, wherein the composition comprises 50 mg to 20,000 mg of said betaine compound.

27. The method according to claim 25, wherein the composition comprises 500 mg to 2000 mg of said betaine compound.

28. The method of claim 16, wherein said reduced folate compound is 5-methyl-tetrahydrofolate.

29. The method of claim 16, wherein said reduce folate compound is 5-formyl tetra hydrofolate.

30. The method of claim 16, wherein said reduced folate is selected from the group consisting of (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, and 5-formimino-(6S)-tetrahydrofolic acid.

31. The method of claim 16, wherein said reduced folate compound is administered at a dose of 0.1-50 mg/day.

32. The method of claim 16, wherein said reduced folate compound is administered at a dose of 0.1-5 mg/day.

33. The method of claim 21, wherein said cobalamin compound is administered at a dose of 0.002-1 mg/day.

34. The method of claim 25, wherein said betaine compound is administered at a dose of 500-2000 mg/day.
